SEQUENCE LISTING (10 sequences)

SEQ ID NO: 1 - DNA, 30 bp, synthetic construct
  source 1..30: mol_type = other DNA; note = P7 with an 8oxoG linearization site
  misc_feature 26: note = 8oxoG linearization site
  modified_base 1: mod_base = OTHER; note = 5Hexynyl
  modified_base 26: mod_base = OTHER; note = 8-Oxoguanine
ttttttcaagcagaagacggcatacgagat

SEQ ID NO: 2 - DNA, 35 bp, synthetic construct
  source 1..35: mol_type = other DNA; note = P5 with a U linearization site
  modified_base 1: mod_base = OTHER; note = 5Hexynyl
  misc_feature 29: note = U linearization site
  modified_base 29: mod_base = OTHER; note = uracil
ttttttaatgatacggcgaccaccgagatctacac

SEQ ID NO: 3 - DNA, 35 bp, synthetic construct; identical to SEQ ID NO: 2
  source 1..35: mol_type = other DNA; note = P15 with a vinyl dT linearization site
  modified_base 1: mod_base = OTHER; note = 5Hexynyl
  misc_feature 29: note = vinyl dT linearization site
  modified_base 29: mod_base = OTHER; note = vinyl dThymidine

SEQ ID NO: 4 - DNA, 35 bp, synthetic construct; identical to SEQ ID NO: 2
  source 1..35: mol_type = other DNA; note = BCN P15 with a vinyl dT linearization site
  modified_base 1: mod_base = OTHER; note = Bicyclononyne
  misc_difference 29: note = vinyl dT linearization site
  modified_base 29: mod_base = OTHER; note = vinyl dThymidine

SEQ ID NO: 5 - DNA, 30 bp, synthetic construct; identical to SEQ ID NO: 1
  source 1..30: mol_type = other DNA; note = BCN P7 with an 8oxoG linearization site
  modified_base 1: mod_base = OTHER; note = Bicyclononyne
  misc_difference 26: note = 8oxoG linearization site
  modified_base 26: mod_base = OTHER; note = 8-Oxoguanine

SEQ ID NO: 6 - DNA, 30 bp, synthetic construct
  source 1..30: mol_type = other DNA; note = P7 with a U linearization site
  modified_base 1: mod_base = OTHER; note = 5Hexynyl
  misc_difference 26: note = U linearization site
  modified_base 26: mod_base = OTHER; note = uracil
ttttttcaagcagaagacggcatactagat

SEQ ID NO: 7 - DNA, 35 bp, synthetic construct
  source 1..35: mol_type = other DNA; note = P5 with an 8oxoG linearization site
  modified_base 1: mod_base = OTHER; note = 5Hexynyl
  misc_difference 29: note = 8oxoG linearization site
  modified_base 29: mod_base = OTHER; note = 8-Oxoguanine
ttttttaatgatacggcgaccaccgagagctacac

SEQ ID NO: 8 - DNA, 35 bp, synthetic construct; identical to SEQ ID NO: 7
  source 1..35: mol_type = other DNA; note = P15 with an 8oxoG linearization site
  modified_base 1: mod_base = OTHER; note = 5Hexynyl
  misc_difference 29: note = 8oxoG linearization site
  modified_base 29: mod_base = OTHER; note = 8-Oxoguanine

SEQ ID NO: 9 - DNA, 35 bp, synthetic construct; identical to SEQ ID NO: 7
  source 1..35: mol_type = other DNA; note = BCN P15 with an 8oxoG linearization site
  modified_base 1: mod_base = OTHER; note = Bicyclononyne
  misc_difference 29: note = 8oxoG linearization site
  modified_base 29: mod_base = OTHER; note = 8-Oxoguanine

SEQ ID NO: 10 - DNA, 31 bp, synthetic construct
  source 1..31: mol_type = other DNA; note = BCN P7 with a vinyl dT linearization site
  modified_base 1: mod_base = OTHER; note = Bicyclononyne
  misc_difference 26: note = vinyl dT linearization site
  modified_base 26: mod_base = OTHER; note = vinyl dThymidine
ttttttcaagcagaagacggcatacttagat

(19) United States
(12) Patent Application Publication
(10) Pub. No.: US 2024/0409994 A1
(43) Pub. Date: Dec. 12, 2024

(54) METHODS OF SEQUENCING DNA USING FLOW CELLS CONTAINING MULTIPLE SURFACES

(71) Applicant: Illumina, Inc., San Diego, CA (US)

(72) Inventor: Thomas Gros, Encinitas, CA (US)

(73) Assignee: Illumina, Inc., San Diego, CA (US)

(21) Appl. No.: 18/731,773

(22) Filed: Jun. 3, 2024

Related U.S. Application Data
(60) Provisional application No. 63/507,402, filed on Jun. 9, 2023.

(51) Int. Cl. C12Q 1/6869

(52) U.S. Cl. CPC C12Q 1/6869

(57) ABSTRACT

Disclosed herein are flow cells and methods of sequencing using flow cells. In some examples, the flow cells include a first surface that includes a first set of capture primers and a second set of capture primers, and a second surface that includes a first set of capture primers and a second set of capture primers.

### CROSS-REFERENCE TO RELATED APPLICATIONS

This application claims the benefit of U.S. Provisional Patent Application No. 63/507,402, filed Jun. 9, 2023 and entitled “Methods of Sequencing DNA Using Flow Cells Containing Multiple Surfaces,” the entire contents of which are incorporated by reference herein.

### INCORPORATION BY REFERENCE OF SEQUENCE LISTING

The material in the accompanying sequence listing is hereby incorporated by reference into the application. The accompanying sequence listing XML file, named “IP-2436-US.xml”, was created on Jun. 5, 2024 and is 28 kB in size.

### FIELD

This application relates to sequencing DNA using flow cells.

### BACKGROUND

During sequencing reactions of DNA, as a sequencing read moves further way from the point of origin, the sequencing reaction becomes less reliable. To address this issue, sequencing reactions often include two sequencing reads, in which the first read sequences DNA in one direction, which is followed by a second read that sequences the DNA in the opposite direction. However, sequencing reactions that require two consecutive sequencing reads can be time consuming and costly.

### SUMMARY

Some examples herein provide a method of sequencing DNA in a flow cell, including binding the DNA to a first set of capture primers and to a second set of capture primers, wherein the first set of capture primers and the second set of capture primers are each coupled to a first surface and to a second surface of the flow cell, wherein the first set of capture primers on the first surface and the second set of capture primers on the second surface each include a first linearization moiety, and wherein the second set of capture primers on the first surface and the first set of capture primers on the second surface each include a second linearization moiety; linearizing the first linearization moiety or the second linearization moiety, resulting in the DNA remaining coupled to either (i) the first set of capture primers on the first surface and the second set of capture primers on the second surface, or (ii) the second set of capture primers on first surface and first set of capture primers on the second surface; coupling the DNA bound to either (i) or (ii) with one or more sequencing primers; and performing at least one sequencing read on the bound DNA, using the one or more sequencing primers.

In some examples, the first surface includes a top surface of the flow cell and the second surface includes a bottom surface of the flow cell.

In some examples, the DNA includes a first nucleotide sequence on a first end of the DNA that is sufficiently complementary to the first set of capture primers such that the first end and the first set of capture primers are capable of binding to each other, and the first nucleotide sequence lacks sufficient complementarity with the second set of capture primers, such that the first nucleotide sequence does not bind to the second set of capture primers.

In some examples, the DNA includes a second nucleotide sequence on a second end of the DNA that is sufficiently complementary to the second set of capture primers such that the second end of the DNA and the second set of capture primers are capable of binding to each other, and the second nucleotide sequence lacks sufficient complementarity to the first set of capture primers, such that the second nucleotide sequence does not bind to the first set of capture primers.

In some examples, the first nucleotide sequence and the second nucleotide sequence is on a positive strand of DNA. In some examples, the first nucleotide sequence and the second nucleotide sequence is on a negative strand of DNA.

In some examples, the method of sequencing DNA further includes at least one step in which the DNA is amplified in the flow cell. In some examples, the DNA is amplified through bridge amplification or exclusion amplification.

In some examples, binding the DNA to a first set of capture primers and to a second set of capture primers, includes binding a plurality of positive strands and a plurality of negative strands to the first set of capture primers and the second set of capture primers.

In some examples, performing the at least one sequencing read includes performing a first sequencing read and a second sequencing read, wherein the first sequencing read is performed on the plurality of positive strands on the first surface and the second sequencing read is performed on the plurality of negative strands on the second surface. In some examples, performing the at least one sequencing read includes performing a first sequencing read and a second sequencing read, wherein the first sequencing read is performed on the plurality of negative strands on the first surface and the second sequencing read is performed on the plurality of positive strands on the second surface.

Some examples herein provide a flow cell, including a first surface including a first set of capture primers and a second set of capture primers; and a second surface including a first set of capture primers and a second set of capture primers, wherein the first set of capture primers on the first surface and the second set of capture primers on the second surface each include a first linearization moiety, and wherein the second set of capture primers on the first surface and the first set of capture primers on the second surface each include a second linearization moiety.

In some examples, the first surface includes a top surface of the flow cell and the second surface includes a bottom surface of the flow cell. In some examples, the first linearization moiety includes any one or more of 8oxoG, U, and vinyl dT, and the second linearization moiety includes any one or more of 8oxoG, U, and vinyl dT.

In some examples, the first set of capture primers includes at least 80% identity with any one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. In some examples, the first set of capture primers includes any one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

In some examples, (i) when the first set of capture primers on the first surface includes at least 80% sequence identity with SEQ ID NO: 1, the first set of capture primers on the second surface includes at least 80% sequence identity with SEQ ID NO: 6, (ii) when the first set of capture primers on the first surface includes at least 80% sequence identity with SEQ ID NO: 2, the first set of capture primers on the second surface includes at least 80% sequence identity with SEQ ID NO: 7, (iii) when the first set of capture primers on the first surface includes at least 80% sequence identity with SEQ ID NO: 3, the first set of capture primers on the second surface includes at least 80% sequence identity with SEQ ID NO: 8, (iv) when the first set of capture primers on the first surface includes at least 80% sequence identity with SEQ ID NO: 4, the first set of capture primers on the second surface includes at least 80% sequence identity with SEQ ID NO: 9, or (v) when the first set of capture primers on the first surface includes at least 80% sequence identity with SEQ ID NO: 5, the first set of capture primers on the second surface includes at least 80% sequence identity with SEQ ID NO: 10. In some examples, (i) the first set of capture primers on the first surface includes SEQ ID NO: 1 and the first set of capture primers on the second surface includes SEQ ID NO: 6, (ii) the first set of capture primers on the first surface includes SEQ ID NO: 2 and the first set of capture primers on the second surface includes SEQ ID NO: 7, (iii) the first set of capture primers on the first surface includes SEQ ID NO: 3 and the first set of capture primers on the second surface includes SEQ ID NO: 8, (iv) the first set of capture primers on the first surface includes SEQ ID NO: 4 and the first set of capture primers on the second surface includes SEQ ID NO: 9, or (v) the first set of capture primers on the first surface includes SEQ ID NO: 5 and the first set of capture primers on the second surface includes SEQ ID NO: 10.

In some examples, the second set of capture primers includes at least 80% identity with any one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. In some examples, the second set of capture primers includes any one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

In some examples, (i) when the second set of capture primers on the first surface includes at least 80% sequence identity with SEQ ID NO: 1, the second set of capture primers on the second surface includes at least 80% sequence identity with SEQ ID NO: 6, (ii) when the second set of capture primers on the first surface includes at least 80% sequence identity with SEQ ID NO: 2, the second set of capture primers on the second surface includes at least 80% sequence identity with SEQ ID NO: 7, (iii) when the second set of capture primers on the first surface includes at least 80% sequence identity with SEQ ID NO: 3, the second set of capture primers on the second surface includes at least 80% sequence identity with SEQ ID NO: 8, (iv) when the second set of capture primers on the first surface includes at least 80% sequence identity with SEQ ID NO: 4, the second set of capture primers on the second surface includes at least 80% sequence identity with SEQ ID NO: 9, or (v) when the second set of capture primers on the first surface includes at least 80% sequence identity with SEQ ID NO: 5, the second set of capture primers on the second surface includes at least 80% sequence identity with SEQ ID NO: 10. In some examples (i) the second set of capture primers on the first surface includes SEQ ID NO: 1 and the second set of capture primers on the second surface includes SEQ ID NO: 6, (ii) the second set of capture primers on the first surface includes SEQ ID NO: 2 and the second set of capture primers on the second surface includes SEQ ID NO: 7, (iii) the second set of capture primers on the first surface includes SEQ ID NO: 3 and the second set of capture primers on the second surface includes SEQ ID NO: 8, (iv) the second set of capture primers on the first surface includes SEQ ID NO: 4 and the second set of capture primers on the second surface includes SEQ ID NO: 9, or (v) the second set of capture primers on the first surface includes SEQ ID NO: 5 and the second set of capture primers on the second surface includes SEQ ID NO: 10.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1A schematically illustrates an example of a positive and a negative strand of DNA linked to adaptor sequences on each end of the positive strand and negative strand.

FIG. 1B schematically illustrates an example of a flow cell containing a first surface and a second surface in which capture primers are bound to each of the first surface and the second surface.

FIG. 2 schematically illustrates an example of a sequencing reaction using the flow cell depicted in FIG. 1B.

FIG. 3 schematically illustrates an example of a sequencing reaction using the flow cell depicted in FIG. 1B.

FIG. 4 schematically illustrates an example of DNA that is processed to include A overhangs and adaptors bound to each end of the DNA.

FIG. 5 schematically illustrates an example of processing DNA to prepare it for amplification and sequencing.

FIGS. 6 and 7 schematically illustrate an example of sequencing positive strand DNA and negative strand DNA in a flow cell.

FIG. 8 schematically illustrates an example of sequencing negative strand DNA in a flow cell.

FIG. 9 schematically illustrates an example of sequencing negative strand DNA from both ends of the negative strand.

FIG. 10 schematically illustrates an example of sequencing positive strand DNA from both ends of the positive strand.

FIGS. 11A and 11B schematically illustrate an example of a double-stranded template library, and seeding of the library onto a flow cell such that it binds to the P5 and P7 capture primers on a flow cell with a single surface (FIG. 11A) or a flow cell with first surface and a second surface (FIG. 11B).

### DETAILED DESCRIPTION

Sequencing DNA using two consecutive reads in which the first read sequences the DNA in one direction followed by the second read that sequences the DNA in the opposite direction, can be time consuming and costly. In addition, sequencing a DNA strand becomes less reliable and more prone to sequencing errors, as the sequencing read proceeds from the point of origin. Described herein are examples of sequencing DNA that addresses these issues. In some examples, each strand of DNA is sequenced using a single read, to reduce costs and reduce sequencing time. In some examples, to limit sequencing errors, each end of each strand of DNA is sequenced using a single read. In some examples, the DNA strands are sequenced on surfaces of flow cells.

Examples provided herein are related to flow cells containing a first surface and a second surface, and sequencing DNA in flow cells containing a first surface and a second surface. In some examples, the first surface is a top surface of the flow cell and the second surface is the bottom surface of the flow cell. In some examples, the first surface and the second surface each contain a first set of capture primers and a second set of capture primers. In some examples, the first set of capture primers on the first surface and the second set of capture primers on the second surface each contain a first linearization moiety. In some examples, the second set of capture primers on the first surface and the first set of capture primers on the second surface each contain a second linearization moiety. In some examples, the linearization profile of the capture primers in the flow cell, allow for each strand of DNA to be read from each end of the strand.

In some examples, after cutting either the first linearization moiety or the second linearization moiety, strands of DNA are sequenced in both directions. In some examples, positive strands of DNA are sequenced in both directions. In some examples, negative strands of DNA are sequenced in both directions. In some examples, both positive strands and negative strands are sequenced in both directions.

First, some terms used herein will be briefly explained. Then, some example compositions and example methods using the compositions will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to #1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes disassociate from one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 5-halouracil, 5-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “primer” is defined as a polynucleotide to which nucleotides may be added via a free 3′ OH group. A primer may include a 3′ block preventing polymerization until the block is removed. A primer may include a modification at the 5′ terminus to allow a coupling reaction or to couple the primer to another moiety. A primer may include one or more moieties which may be cleaved under suitable conditions, such as UV light, chemistry, enzyme, or the like. The primer length may be any suitable number of bases long and may include any suitable combination of natural and non-natural nucleotides. A target polynucleotide may include an “adaptor” that hybridizes to (has a sequence that is complementary to) a primer, and may be amplified so as to generate a complementary copy polynucleotide by adding nucleotides to the free 3′ OH group of the primer.

A “sequencing primer” is intended to mean a primer capable of binding to a DNA template strand or to an adaptor connected to the DNA template strand, that is capable of carrying out a sequencing reaction.

A “capture primer” is intended to mean a primer that is coupled to a flow cell and may hybridize to an adaptor of the target polynucleotide. The adaptors may have respective sequences that are complementary to those of capture primers to which they may hybridize. A capture primer that may be used to hybridize to an adaptor of a target polynucleotide in order to couple that polynucleotide to the flow cell, but that may not be used to grow a complementary strand during an amplification process, may in some cases be referred to as a “seeding primer.” A capture primer that may be used to grow a complementary strand during an amplification process may in some cases be referred to as an “amplification primer”.

As used herein, the phrase “linearization moiety” refers to any structural feature on a nucleic acid that is capable of being cut by enzyme. A non-limiting list of “linearization moieties” include 8oxoG, U, and vinyl dT.

### Compositions for Sequencing

Some examples of compositions provided herein include DNA that is attached to adaptor sequences. In some examples, each strand of the DNA is attached to adaptor sequences. In some examples, each end of the positive strand is attached to an adaptor sequence. In some examples, each end of the negative strand is attached to an adaptor sequence.

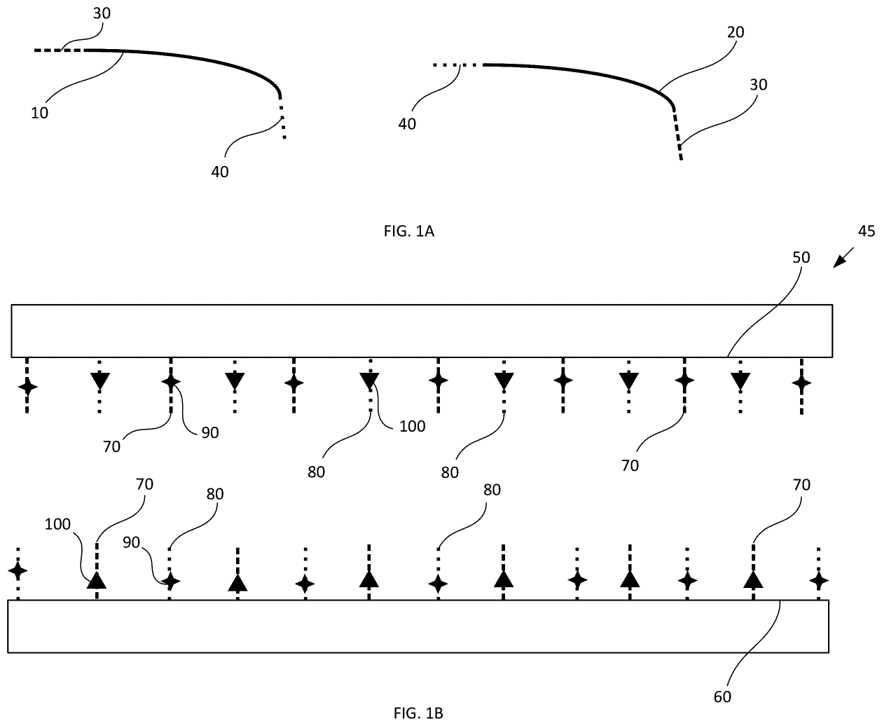

In a non-limiting example, FIG. 1A illustrates a positive strand of DNA 10 and a negative strand of DNA 20, with each end attached to adaptor sequences. In some examples, a first adaptor sequence 30 is linked to the 5′ end of the positive strand and the negative strand. In some examples, a second adaptor sequence 40 is linked to the 3′ end of the positive strand and the negative strand. In some examples, the first adaptor sequence 30 is linked to the 3′ end of the positive strand and negative strand. In some examples, the second adaptor sequence 40 is linked to the 5′ end of the positive strand and the negative strand.

In some examples, the positive strand of DNA 10 and the negative strand of DNA 20, illustrated in FIG. 1A, each derive from the same DNA. In some examples, the positive strand of DNA 10 and the negative strand of DNA 20, illustrated in FIG. 1A, each derive from different DNA.

In some examples, prior to linking the adaptor sequences to the positive strands and negative strands, the positive strand and negative strand are processed to create a nucleotide overhang on one end of the positive strand and on one end of the negative strand. In some examples, the adaptor sequences are oriented such that the first adaptor sequence is linked to the end of the positive strand and the end of the negative strand that contain the nucleotide overhang. In some examples, the adaptor sequences are oriented such that the second adaptor sequence is linked to the end of the positive strand and the end of the negative strand that contain the nucleotide overhang.

Some examples of compositions described herein include flow cells that contain a first surface and a second surface, wherein capture primers are attached to the first surface and the second surface.

Flow cells may include a flow chamber that is divided into a plurality of lanes or a plurality of sectors. Example flow cells and substrates for manufacture of flow cells that may be used in methods and compositions set forth herein include, but are not limited to, those commercially available from Illumina, Inc. (San Diego, CA).

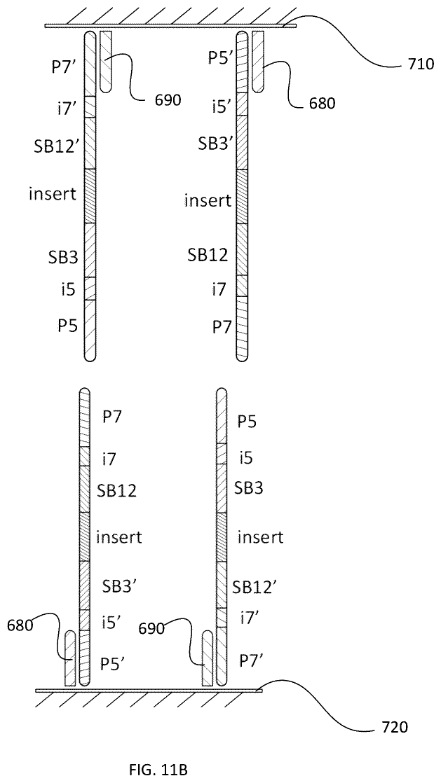

In some examples, the first surface of the flow cell includes a top surface. In some examples, the second surface of the flow cell includes a bottom surface. A non-limiting example of a flow cell containing a top surface and a bottom surface, is illustrated in FIG. 1B. Specifically, FIG. 1B illustrates a flow cell 45 that contains a first surface 50 and a second surface 60. As illustrated in FIG. 1B, the first surface includes 50 a top surface and the second surface 60 includes a bottom surface. Bound to each of the top surface and bottom surface are first capture primers 70 and second capture primers 80. The first capture primers 70 contain nucleotide regions that are at least partially complementary to at least a portion of the first adaptor sequence (element 30 shown in FIG. 1A). The first capture primers lack sufficient complementarity to bind to the second adaptor sequence (element 40 shown in FIG. 1A). The second capture primers 80 contain nucleotide regions that are at least partially complementary to at least a portion of the second adaptor sequence (element 40 shown in FIG. 1A). The second capture primers lack sufficient complementarity to bind to the first adaptor sequence (element 30 shown in FIG. 1A). As described herein and shown in FIGS. 11A and 11B, the first adaptor sequence and the second adaptor can include other elements such as an index sequence and a sequencing binding site.

In some examples, any of the capture primers used in the flow cells described herein, include at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. In some examples, any of the capture primers herein may include any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

In some examples, each of the first capture primers and second capture primers contain a linearization moiety. As shown in FIG. 1B, on the first surface 50, the first capture primers 70 contain a first linearization moiety 90, and the second capture primers 80 contain a second linearization moiety 100. On the second surface, the first capture primers 70 contain the second linearization moiety 100, and the second capture 80 primers contain the first linearization moiety 90. The linearization moiety 90 and the linearization moiety 100 may be cleaved using different enzymes.

In some examples, on the first surface, the first capture primers contain the second linearization moiety, and the second capture primers contain the first linearization moiety. In some examples, on the second surface, the first capture primers contain the second linearization moiety, and the second capture primers contain the first linearization moiety.

In some examples, the first linearization moiety can by any of vinyl dT, 8oxoG, or U, and the second linearization moiety can be a different one of vinyl dT, 8oxoG, or U. And, it should be understood that the first linearization moiety and the second linearization moiety can be of the other types of moieties that are different from one another. In some examples, the first linearization moiety and the second linearization moiety include any moiety that is a structural featural on a nucleic acid (e.g., DNA) capable of being cut by an enzyme.

Some examples herein provide a flow cell including a first surface including a first set of capture primers and a second set of capture primers; and a second surface including the first set of capture primers and the second set of capture primers, wherein the first set of capture primers on the first surface and the second set of capture primers on the second surface each include a first linearization moiety, and wherein the second set of capture primers on the first surface and the first set of capture primers on the second surface each include a second linearization moiety.

In some examples, the first surface includes a top surface of the flow cell and the second surface includes a bottom surface of the flow cell. In some examples, the first surface and the second surface are in the same plane on the flow cell. In some examples, the first surface of the flow cell and the second surface of the flow cell are on planes that are perpendicular to each other.

In some examples, the first set of capture primers includes at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with any one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

In some examples, the first set of capture primers includes any one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

In some examples, the second set of capture primers includes at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity with any one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

In some examples, the second set of capture primers include any one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

In some examples, when a first set of capture primers on a first surface of a flow cell are any of the percent identities described herein of SEQ ID NO: 1, then a first set of capture primers on a second surface of the flow cell are any of the percent identities described herein of SEQ ID NO: 6. In some examples, when a first set of capture primers on a first surface of a flow cell are any of the percent identities described herein of SEQ ID NO: 2, then a first set of capture primers on a second surface of the flow cell are any of the percent identities described herein of SEQ ID NO: 7. In some examples, when a first set of capture primers on a first surface of a flow cell are any of the percent identities described herein of SEQ ID NO: 3, then a first set of capture primers on a second surface of the flow cell are any of the percent identities described herein of SEQ ID NO: 8. In some examples, when a first set of capture primers on a first surface of a flow cell are any of the percent identities described herein of SEQ ID NO: 4, then a first set of capture primers on a second surface of the flow cell are any of the percent identities described herein of SEQ ID NO: 9. In some examples, when a first set of capture primers on a first surface of a flow cell are any of the percent identities described herein of SEQ ID NO: 5, then a first set of capture primers on a second surface of the flow cell are any of the percent identities described herein of SEQ ID NO: 10.

In some examples, when a second set of capture primers on a first surface of a flow cell are any of the percent identities described herein of SEQ ID NO: 1, then a second set of capture primers on a second surface of the flow cell are any of the percent identities described herein of SEQ ID NO: 6. In some examples, when a second set of capture primers on a first surface of a flow cell are any of the percent identities described herein of SEQ ID NO: 2, then a second set of capture primers on a second surface of the flow cell are any of the percent identities described herein of SEQ ID NO: 7. In some examples, when a second set of capture primers on a first surface of a flow cell are any of the percent identities described herein of SEQ ID NO: 3, then a second set of capture primers on a second surface of the flow cell are any of the percent identities described herein of SEQ ID NO: 8. In some examples, when a second set of capture primers on a first surface of a flow cell are any of the percent identities described herein of SEQ ID NO: 4, then a second set of capture primers on a second surface of the flow cell are any of the percent identities described herein of SEQ ID NO: 9. In some examples, when a second set of capture primers on a first surface of a flow cell are any of the percent identities described herein of SEQ ID NO: 5, then a second set of capture primers on a second surface of the flow cell are any of the percent identities described herein of SEQ ID NO: 10.

### Methods of Sequencing

Some examples herein provide methods of sequencing using any of the flow cells described herein. In some examples, the flow cells used for sequencing contain a first set of capture primers and a second of capture primers, with each of the first capture primers and the second capture primers containing linearization moieties.

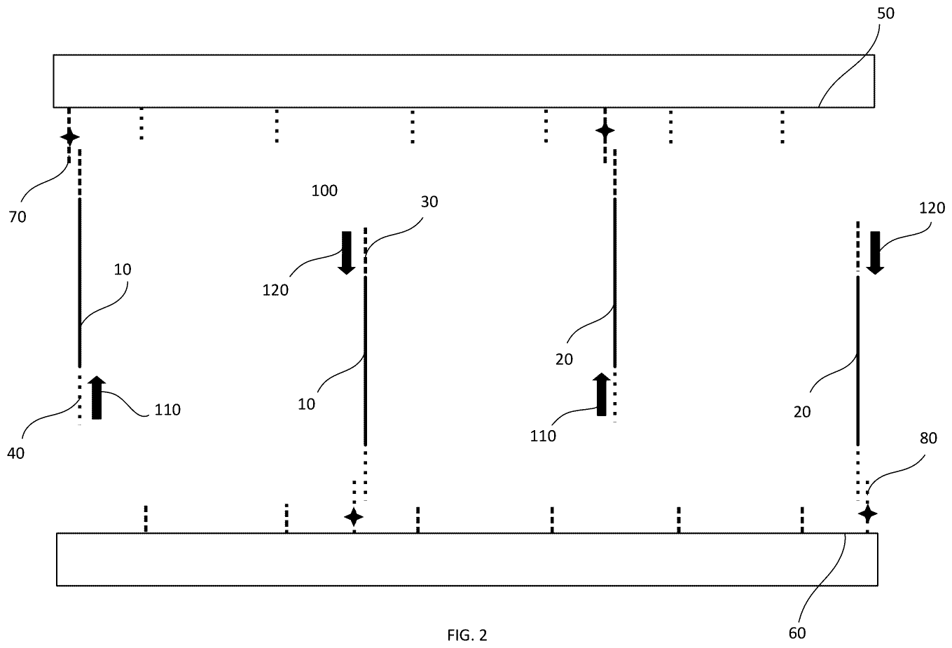

A non-limiting example of a sequencing reaction using the positive strand of DNA 10 (FIG. 1A), the negative strand of DNA 20 (FIG. 1A) and the flow cell 45 (FIG. 1B), is illustrated in FIG. 2. DNA can be amplified by any method described herein, thus, creating multiple copies of the positive strands 10 (FIG. 1A) and negative strands 20 (FIG. 1A) that can be sequenced in the flow cell 45. Also, the positive strands of DNA 10 (FIG. 1A) and the negative strands of DNA 20 (FIG. 1A) can derive from the same DNA or different DNA.

After linearization of the second linearization moiety 100 (FIG. 1B), the positive strands 10 and negative strands 20 are captured on the flow cell by the first set of capture primers 70, on the first surface 50. Also, the positive strands and negative strands are captured on the flow cell by the second set of capture primers 80, on the second surface 60. After linearization, sequencing primers 110 bind to at least a portion of the second adaptor sequences 40 (see second adaptor sequences shown in FIG. 1A), and sequencing primers 120 bind to at least a portion of the first adaptor sequences 30 (see first adaptor sequences shown in FIG. 1A). In this example, the sequencing primers 110 can be any sequence that is capable of binding to at least a portion of the second adaptor sequences 40. In this example, the sequencing primers 120 can be any sequence that is capable of binding to at least a portion of the first adaptor sequences 30.

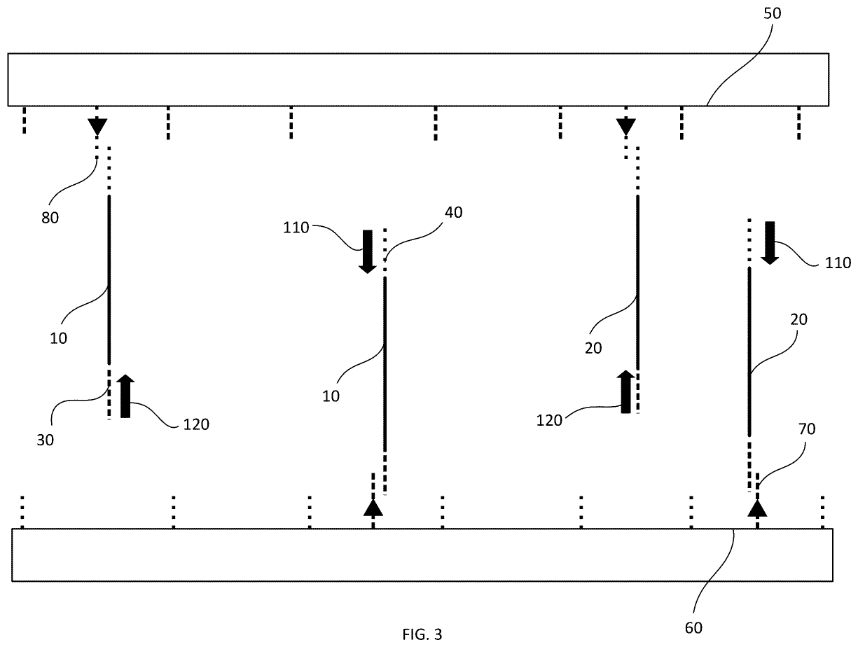

An alternative non-limiting example of a sequencing reaction is shown in FIG. 3. Preceding the sequencing reaction shown in FIG. 3, linearization is performed at the first linearization moiety 90 (FIG. 1B). DNA can be amplified by any method described herein, thus, creating multiple copies of the positive strands 10 (FIG. 1A) and the negative strands 20 (FIG. 1A) that can be sequenced in the flow cell 45

After linearization of the first linearization moiety 90 (FIG. 1B), the positive strands 10 and negative strands 20 are captured the flow cell by the second set of capture primers 80, on the first surface 50. Also, the positive strands and negative strands captured on the flow cell by the first set of capture primers 70, on the second surface 60. Sequencing primers 110 bind to at least a portion of the second adaptor sequences 40 (see second adaptor sequences shown in FIG. 1A), and sequencing primers 120 bind to at least a portion of the first adaptor sequences 30 (see first adaptor sequences shown in FIG. 1B). In this example, the sequencing primers 110 can be any sequence that is capable of binding to at least a portion of the second adaptor sequences 40. In this example, the sequencing primers 120 can be any sequence that is capable of binding to at least a portion of the first adaptor sequences 30.

In some examples, the capture primers 70, 80 (FIG. 1B) on the first surface of the flow cell 50 (FIG. 1B) are P5 and P7 capture primers. In some examples, the capture primers 70, 80 (FIG. 1B) on the second surface of the flow cell 60 (FIG. 1B) are P5 and P7 capture primers. In some examples, the capture primers 70, 80 (FIG. 1B) on both the first surface 50 (FIG. 1B) and the second surface 60 (FIG. 1B) are P5 and P7 capture primers. In some examples, the P5 and P7 capture primers are any P5 and P7 capture primers described herein.

In some examples the P5 and P7 capture primers each contain a linearization moiety. In some examples the linearization moiety of the P5 primers on the first surface 50 (FIG. 1B) match the linearization moiety of the P7 primers on the second surface 60 (FIG. 1B). In some examples, the linearization moiety of the P7 primers on the first surface 50 (FIG. 1B) match the linearization moiety of the P5 primers on the second surface 60 (FIG. 1B). In some examples the linearization moiety of the P5 primers on the first surface 50 (FIG. 1B) match the linearization moiety of the P7 primers on the second surface 60 (FIG. 1B), and the linearization moiety of the P7 primers on the first surface 50 (FIG. 1B) match the linearization moiety of the P5 primers on the second surface 60 (FIG. 1B).

In some examples, the linearization moiety on the P5 primers includes an 8oxoG linearization moiety. In some examples, the linearization moiety on the P5 primers includes a U linearization moiety. In some examples, the linearization moiety on the P7 primers includes an 8oxoG linearization moiety. In some examples, the linearization moiety on the P7 primers includes a U linearization moiety.

In some examples, on the first surface 50 (FIG. 1B), the P7 primers include an 8oxoG linearization site, and the P5 primers include a U linearization site. In some examples, on the second surface 60 (FIG. 1B), the P7 primers include a U linearization site, and the P5 primers include an 8oxoG linearization site. In some examples, on the first surface 50 (FIG. 1B), the P7 primers include a U linearization site, and the P5 primers include an 8oxoG linearization site. In some examples, on the second surface 60 (FIG. 1B), the P7 primers include an 8oxoG linearization site, and the P5 primers include U linearization site.

In some examples, the capture primers on the first surface 50 (FIG. 1B) of the flow cell are P15 and BCN P15 capture primers. In some examples, the capture primers on the second surface 60 (FIG. 1B) of the flow cell are P15 and BCN P15 capture primers. In some examples, the capture primers on both the first surface 50 (FIG. 1B) and the second surface 60 (FIG. 1B) are P15 and BCN P15 capture primers.

In some examples, the P15 and BCN P7 primers each contain a linearization moiety. In some examples the linearization moiety of the P15 primers on the first surface 50 (FIG. 1B) match the linearization moiety of the BCN P7 primers on the second surface 60 (FIG. 1B). In some examples, the linearization moiety of the BCN P7 primers on the first surface 50 (FIG. 1B) match the linearization moiety of the P15 primers on the second surface 60 (FIG. 1B). In some examples, the linearization moiety of the P15 primers on the first surface 50 (FIG. 1B) match the linearization moiety of the BCN P7 primers on the second surface 60 (FIG. 1B), and the linearization moiety of the BCN P7 primers on the first surface 50 (FIG. 1B) match the linearization moiety of the P15 primers on the second surface 60 (FIG. 1B).

In some examples, the linearization moiety on the P15 primers include a vinyl dT linearization moiety. In some examples, the linearization on the P15 primers include an 8oxoG linearization moiety. In some examples, the linearization moiety on the BCN P15 primers includes a vinyl dT linearization moiety. In some examples, the linearization moiety on the BCN P15 primers include an 8oxoG linearization moiety.

In some examples, on the first surface 50 (FIG. 1B), the P15 primers include a vinyl dT linearization site, and the BCN P7 primers include an 8oxoG linearization site. In some examples, on the second surface 60 (FIG. 1B), the P15 primers include an 8oxoG linearization site, and the BCN P7 capture primers include a vinyl dT linearization site. In some examples, on the first surface 50 (FIG. 1B), the P15 primers include an 8oxoG linearization site, and the BCN P7 primers include a vinyl dT linearization site. In some examples, on the second surface 60 (FIG. 1B), the P15 primers include a vinyl dT linearization site, and the BCN P7 primers include an 8oxoG linearization site. In some examples, the P15 primers are substituted with BCN P15 primers.

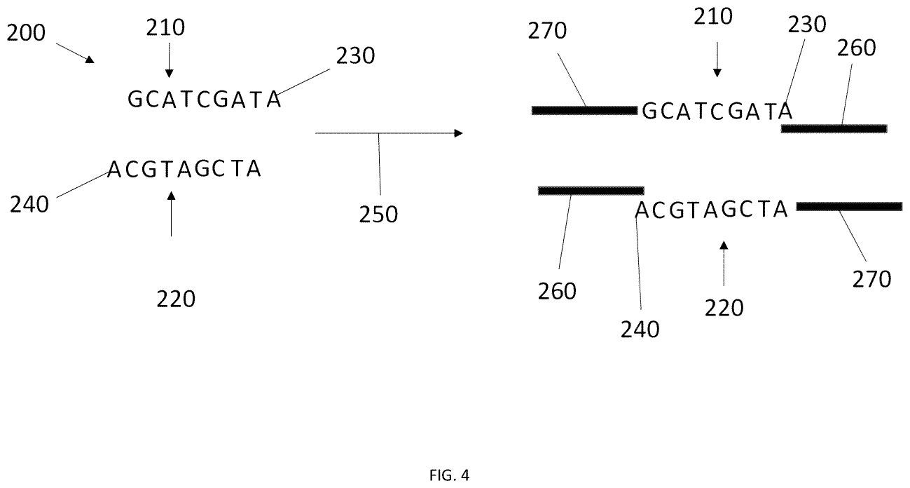

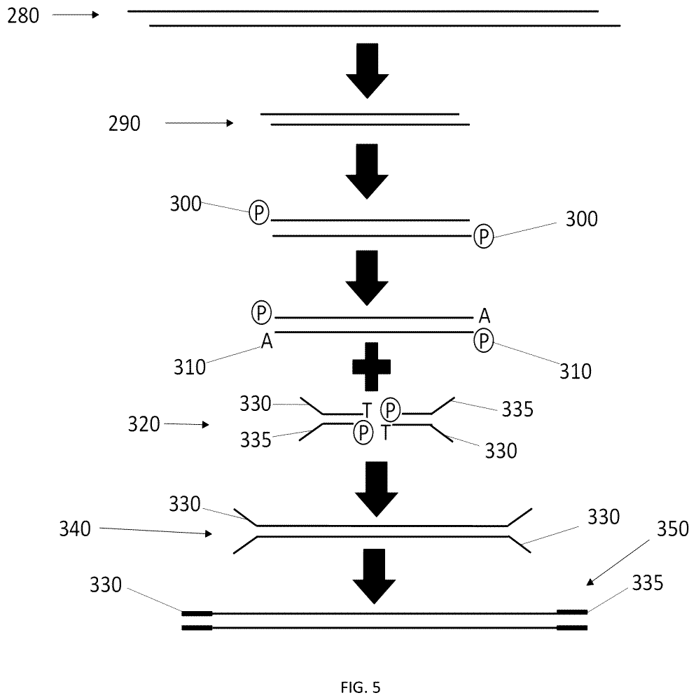

In non-limiting examples, FIGS. 4 and 5 illustrate exemplary methods for preparing DNA samples with adaptor sequences that can be used in any of the sequencing reactions described herein. In some examples, after preparing the DNA samples, the DNA samples are amplified, to create copies of the DNA samples that are then used in any of the sequencing reactions described herein.

FIG. 4 illustrates an example of double stranded DNA molecule 200, which has a positive strand 210 and a negative strand 220. Prior sample processing generated an A nucleotide overhang 230 on one end of the positive strand and an A nucleotide 240 overhang on one end of the negative strand. An additional step of sample processing ligates adaptors to the DNA 250. In some examples, adaptors complementary to P5 primers 260 are added to the end of the DNA strand that contains the A nucleotide overhang. In some examples, adaptors complementary to P7 primers 270 are added to the end of the DNA strand opposite to the location of the P5 adaptors.

FIG. 5 illustrates an example of sample processing to create a DNA product that can be used in amplification and sequencing reactions described herein. Double stranded DNA 280 is fractionated and selected for size using standard techniques 290. The fractionated DNA is repaired through creating blunt ends on the DNA containing phosphorylated groups 300. A nucleotides are added to create an A overhang 310 on the end of the DNA. Adaptors are added 320. Adaptors that contain P5 primer binding sites 330 are added to the side of the DNA that contains the A nucleotide overhang (this is similar to FIG. 4 in which adaptors complementary to P5 primers 260 are added to the end that contains the A nucleotide overhang). Adaptors that contain the P7 primer binding sites are added 335 to the end of the DNA containing the phosphorylated groups. The adaptors are then ligated to the DNA 340. The product can then be used in any of the sequencing reactions described herein. Alternatively, the DNA can then be denatured and amplified 350, and the amplified products can be used in any of the sequencing reactions described herein.

In some examples, amplification of DNA precedes any of the sequencing reactions described herein. In some examples, DNA is amplified using bridge amplification using methods described herein. In some examples, DNA is amplified using exclusion amplification using methods described herein. In some examples, the amplification of DNA utilizes any of the flow cells described herein, for example, a flow cell that includes a first surface and a second surface, and that includes capture primers on both the first and second surface. In some examples, the capture primers include P5 and P7 capture primers that each contain a linearization moiety. In some examples, the linearization moiety of the P5 primers on the first surface match the linearization moiety of the P7 primers on the second surface. In some examples, the linearization moiety of the P7 primers on the first surface match the linearization moiety of the P5 primers on the second surface. In some examples, the linearization moiety of the P5 primers on the first surface match the linearization moiety of the P7 primers on the second surface, and the linearization moiety of the P7 primers on the first surface match the linearization moiety of the P5 primers on the second surface.

In some examples, the amplification methods generate a cluster that can then be sequenced. In some examples, generation of a cluster is performed on both the first surface and the second surface of the flow cell.

In some examples, a flow cell that is used to sequence the positive strands and negative strands of DNA is the same flow cell that is used to amplify the positive strands and negative strands of DNA. In some examples, a flow cell that is used to sequence the positive strands and negative strands of DNA is a different flow than is used to amplify the positive strands and negative strands of DNA.

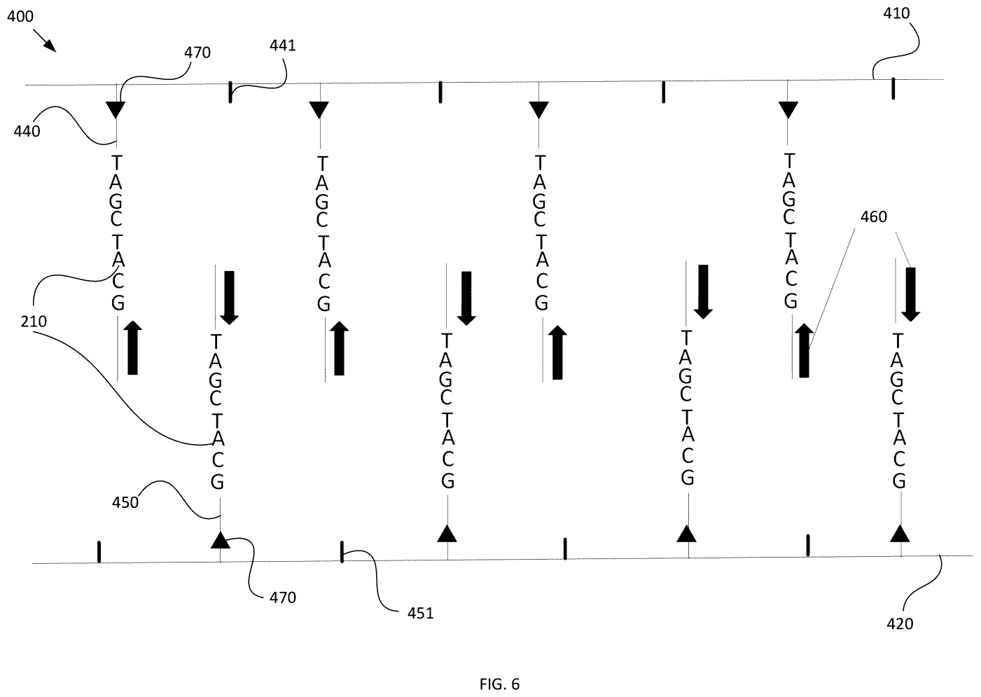

FIG. 6 illustrates an example of a sequencing reaction of positive strands 210 (see FIG. 4) using a flow cell 400 containing a first surface 410 and a second surface 420. After linearization, the positive strands are coupled to P5 primers 440 on the first surface, and the positive strands are coupled to the P7 primers 450 on the second surface. After linearization, a portion of the P7 primers 441 remain on the first surface and a portion of the P5 primers 451 remain on the second surface. Sequencing primers 460 are used to sequence the positive strands. As illustrated in FIG. 6, the positive strands are sequenced in both directions using the sequencing primers.

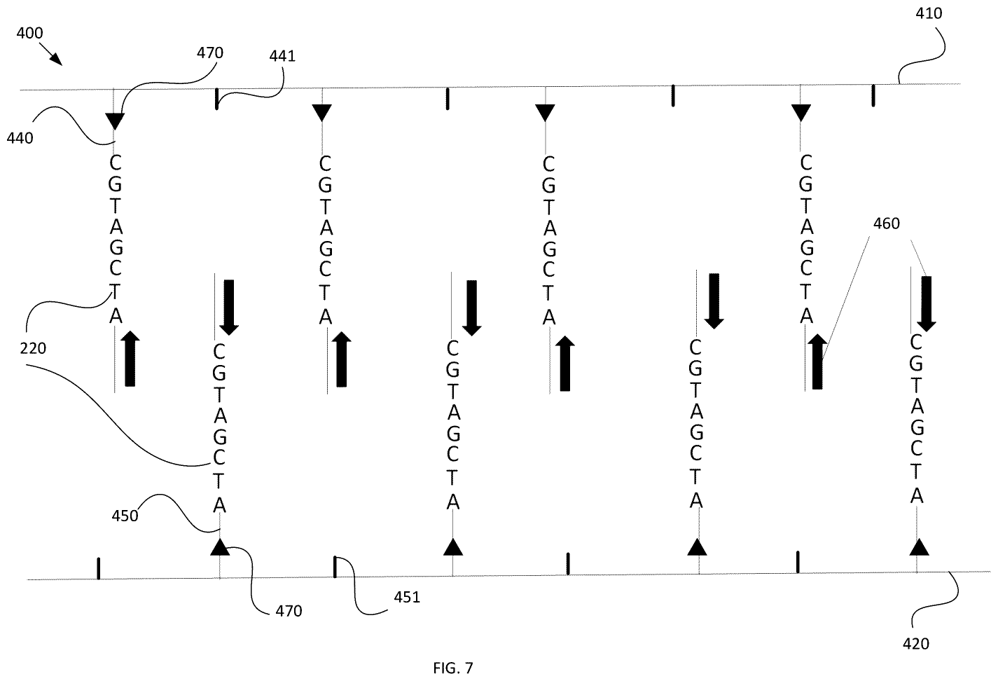

FIG. 7 illustrates a sequencing reaction of negative strands 220 (see negative strand sequence from FIG. 4) using the same flow cell 400 shown in FIG. 6. After linearization, the negative strands are coupled to P5 primers 440 on the first surface, and the negative strands are coupled to the P7 primers 450 on the second surface. After linearization, a portion of the P7 primers 441 remain on the first surface and a portion of the P5 primers 451 remain on the second surface. Sequencing primers 460 are used to sequence the negative strands. As illustrated in FIG. 7, the negative strands are sequenced in both directions using the sequencing primers.

In some examples, a single flow cell is used to sequence the positive strands and the negative strands. Accordingly, the sequencing reactions of the positive strands and negative strands shown in FIGS. 6 and 7 can be performed single flow cell, as illustrated.

In some examples, when a single flow cell is used to sequence the positive strands and the negative strands, the positive strands and the negative strands are sequenced in a specific order. In some examples, the positive strands are sequenced first on the first surface of the flow cell 400. In some examples, the negative strands are sequenced first on the second surface of the flow cell 400. In some examples, the positive strands are sequenced first on the first surface of the flow cell 400, and the negative strands are sequenced first on the second surface of the flow cell 400. In some examples, the positive strands are sequenced first on the second surface of the flow cell 400. In some examples, the negative strands are sequenced first on the first surface of the flow cell 400. In some examples, the positive strands are sequenced first on the second surface of the flow cell 400, and the negative strands are sequenced first on the first surface of the flow cell 400.

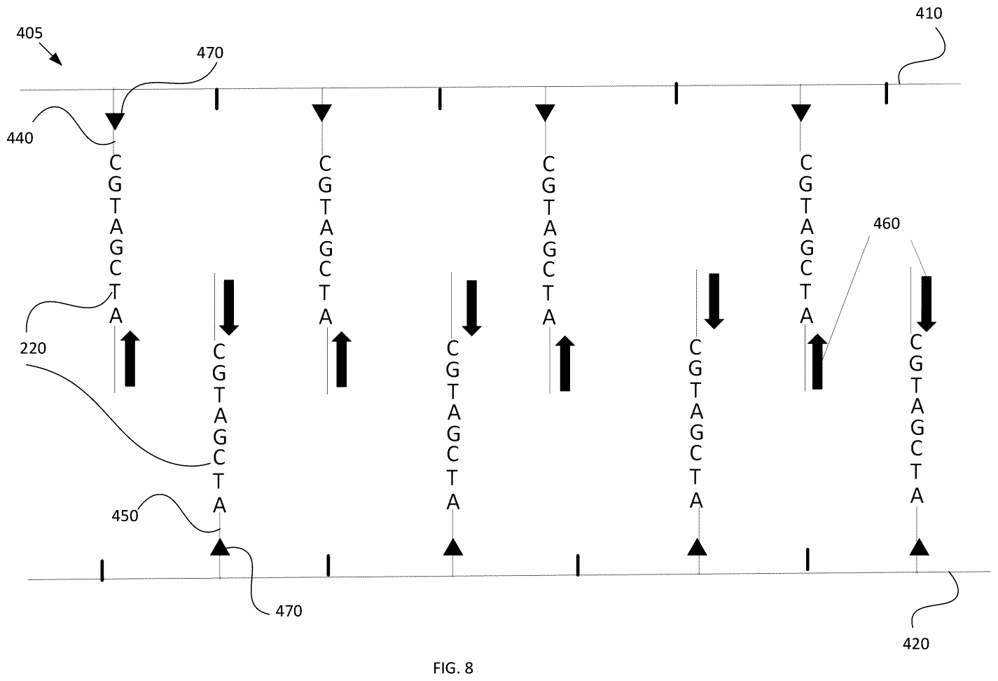

In some examples, the positive strands and negative strands are sequenced in separate flow cells. Thus, in an alternative example, the flow cell 400 illustrated in FIG. 6 is used to sequence the positive strands and the flow cell 405 illustrated in FIG. 8 is used to sequence the negative strands. Note, that the components of the flow cell 400 shown in FIG. 7 are identical to the components of the flow cell 405 shown in FIG. 8.

In some examples, when separate flow cells are used to sequence the positive strands and the negative strands, the positive strands and the negative strands are sequenced in a specific order. In some examples, the positive strands are sequenced first on the first surface of the flow cell 400. In some examples, the negative strands are sequenced first on the second surface of the flow cell 405. In some examples, the positive strands are sequenced first on the first surface of the flow cell 400, and the negative strands are sequenced first on the second surface of the flow cell 405. In some examples, the positive strands are sequenced first on the second surface of the flow cell 400. In some examples, the negative strands are sequenced first on the first surface of the flow cell 405. In some examples, the positive strands are sequenced first on the second surface of the flow cell 400, and the negative strands are sequenced first on the first surface of the flow cell 405.

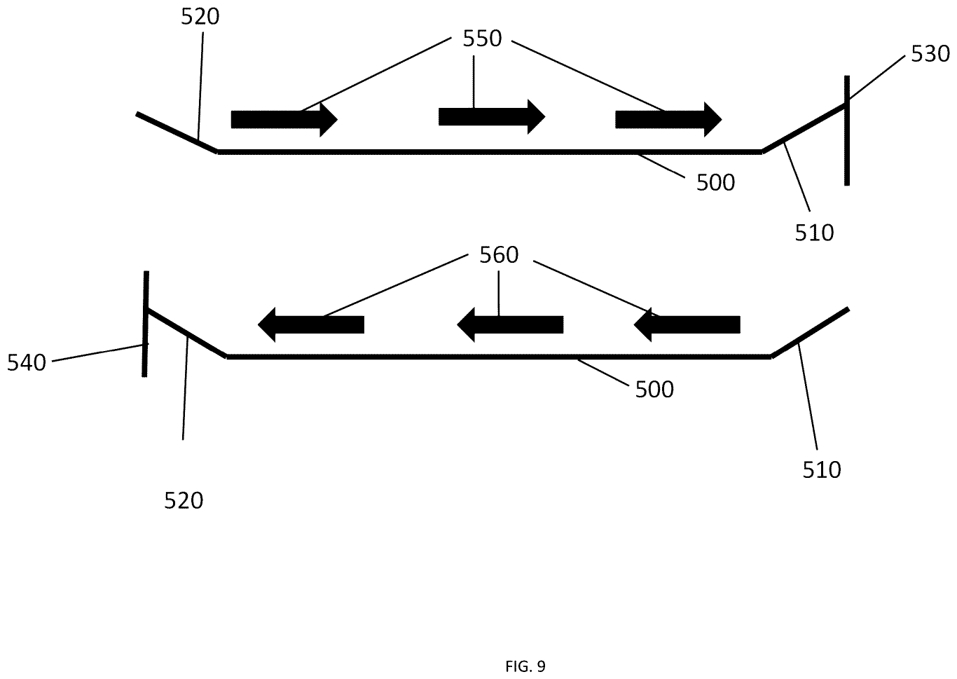

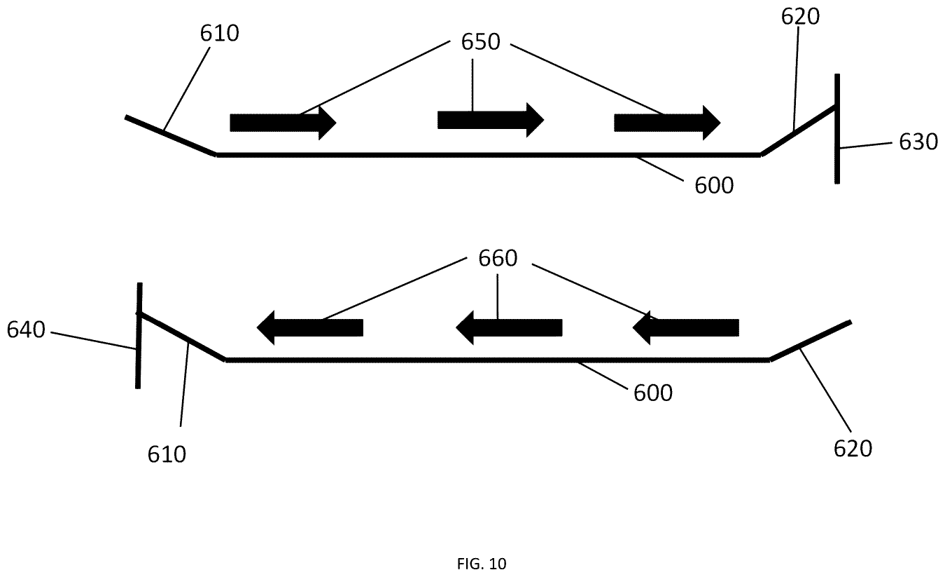

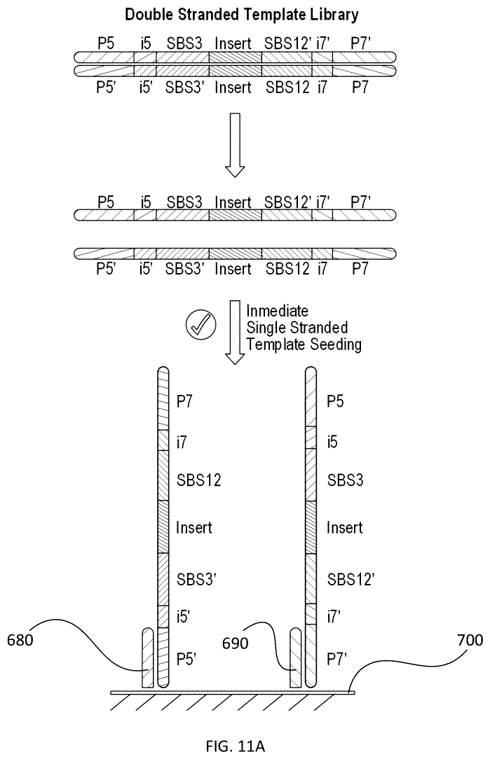

FIGS. 9 and 10 illustrate non-limiting examples of sequencing reactions of a positive strands and negative strands. FIG. 9 illustrates an example of sequencing a negative strand of DNA 500 containing a P5 adaptor sequence 510 and a P7 adaptor sequence 520 on a first surface 530 and a second surface 540. In some examples, the orientation of the P5 adaptor sequences and the P7 adaptor sequences relative to the negative strand can be reversed. Accordingly, the P5 adaptor sequence can be upstream of the direction of sequencing, and the P7 adaptor sequence can be downstream of the direction of sequencing.

As illustrated in FIG. 9, on the first surface, sequencing is performed 550 in the direction towards the first surface. On the second surface, sequencing is performed 560 in the direction towards the second surface. Because sequencing is performed starting at both ends of the negative strand, the sequencing reaction produces a high quality read of the negative strand.

FIG. 10 illustrates an example of sequencing a positive strand of DNA 600 containing a P5 adaptor sequence 610 and a P7 adaptor sequence 620 on a first surface 630 and a second surface 640. In some examples, the orientation of the P5 adaptor sequences and the P7 adaptor sequences relative to the positive strand can be reversed. Accordingly, the P5 adaptor sequence can be upstream of the direction of sequencing, and the P7 adaptor sequence can be downstream of the direction of sequencing.

As illustrated in FIG. 10, on the first surface, sequencing is performed 650 in the direction towards the first surface. On the second surface, sequencing is performed 660 in the direction towards the second surface. Because sequencing is performed starting at both ends of the positive strand, the sequencing reaction produces a high quality read of the positive strand.

Some examples herein provide a method of sequencing DNA in a flow cell, including binding the DNA to a first set of capture primers and to a second set of capture primers, wherein the first set of capture primers and the second set of capture primers are coupled to a first surface and to a second surface of the flow cell, wherein the first set of capture primers on the first surface and the second set of capture primers on the second surface each include a first linearization moiety, and wherein the second set of capture primers on the first surface and the first set of capture primers on the second surface each include a second linearization moiety; linearizing the first linearization moiety or the second linearization moiety, resulting in the DNA remaining coupled to either (i) the first set of capture primers on the first surface and the second set of capture primers on the second surface, or (ii) the second set of capture primers on first surface and first set of capture primers on the second surface; contacting the DNA bound to either (i) or (ii) with one or more sequencing primers; and performing at least one sequencing read on the bound DNA, using the one or more sequencing primers.

In some examples, the first surface includes a top surface of the flow cell and the second surface includes a bottom surface of the flow cell. In some examples, the first surface and the second surface are in the same plane on the flow cell. In some examples, the first surface of the flow cell and the second surface of the flow cell are on planes that are perpendicular to each other.

### Amplification Methods

Any of the sequencing methods described herein can include the additional step of amplification. In some examples, the amplification occurs prior to the sequencing reaction. Any of the sequencing methods described herein can include the additional step of bridge amplification or exclusion amplification.

Bridge Amplification: Bridge amplification can be performed on a flow cell. Single stranded template DNA is hybridised to lawn primers in a flow cell, and a polymerase is used to extend the primer to form double-stranded DNA. The double-stranded DNA is denatured, and the original template strand of the DNA molecule is washed away. This results in a single-stranded DNA molecule being bound to the lawn primers of the flow cell. The single-stranded DNA molecule turns over and forms a “bridge” by hybridising to a nearby lawn primer that is complementary to a sequence of the single-stranded DNA molecule. Polymerase extends the hybridised primer resulting in bridge amplification of the DNA molecule and the creation of a double-stranded DNA molecule. The double-stranded DNA molecule is then denatured resulting in two copies of single-stranded templates, one of which is immobilised to the support and the other of which may be washed away. The one which is immobilised as the support may be used in further bridge amplification operations so as to generate a cluster that subsequently may be sequenced.

The flow cell used in bridge amplification can be any of the flow cells described herein that include a first surface and a second surface, in which each of the first surface and the second surface include P5 and P7 capture primers. In some examples the linearization moiety of the P5 primers on the first surface match the linearization moiety of the P7 primers on the second surface. In some examples, the linearization moiety of the P7 primers on the first surface match the linearization moiety of the P5 primers on the second surface. In some examples, the linearization moiety of the P5 primers on the first surface match the linearization moiety of the P7 primers on the second surface, and the linearization moiety of the P7 primers on the first surface match the linearization moiety of the P5 primers on the second surface.

Exclusion Amplification: Exclusion amplification methods may allow for the amplification of a single target polynucleotide per substrate region and the production of a substantially monoclonal population of amplicons in a substrate region. For example, the rate of amplification of the first captured target polynucleotide within a substrate region may be more rapid relative to much slower rates of transport and capture of target polynucleotides at the substrate region. As such, the first target polynucleotide captured in a substrate region may be amplified rapidly and fill the entire substrate region, thus inhibiting the capture of additional target polynucleotide in the same substrate region. Alternatively, if a second target polynucleotide attaches to same substrate region after the first polynucleotide, the relatively rapid amplification of the first polynucleotide may fill enough of the substrate region to result in a signal that is sufficiently strong to perform sequencing by synthesis (e.g., the substrate region may be at least functionally monoclonal). The use of exclusion amplification may also result in super-Poisson distributions of monoclonal substrate regions; that is, the fraction of substrate regions in an array that are functionally monoclonal may exceed the fraction predicted by the Poisson distribution.

Increasing super-Poisson distributions of useful clusters is useful because more functionally monoclonal substrate regions may result in higher quality signal, and thus improved sequencing by synthesis (SBS); however, the seeding of target polynucleotides into substrate regions may follow a spatial Poisson distribution, where the trade-off for increasing the number of occupied substrate regions is increasing the number of polyclonal substrate regions. One method of obtaining higher super-Poisson distributions is to have seeding occur quickly, followed by a delay among the seeded target polynucleotide. The delay, termed “kinetic delay” because it is thought to arise through the biochemical reaction kinetics, gives one seeded target polynucleotide an earlier start over the other seeded targets. Exclusion amplification works by using recombinase to facilitate the invasion of primers (e.g., primers attached to a substrate region) into double-stranded DNA (e.g., a target polynucleotide) when the recombinase mediates a sequence match. The present compositions and methods may be adapted for use with recombinase to facilitate the invasion of the present capture primers and orthogonal capture primers into the present target polynucleotides when the recombinase mediates a sequence match. Indeed, the present compositions and methods may be adapted for use with any surface-based polynucleotide amplification methods such as thermal PCR, chemically denatured PCR, and enzymatically mediated methods (which may also be referred to as recombinase polymerase amplification (RPA), strand invasion, or ExAmp).

Exclusion amplification can be performed on any of the flow cells described herein that include a first surface and a second surface, in which each of the first surface and the second surface include P5 and P7 capture primers. In some examples the linearization moiety of the P5 primers on the first surface match the linearization moiety of the P7 primers on the second surface. In some examples, the linearization moiety of the P7 primers on the first surface match the linearization moiety of the P5 primers on the second surface. In some examples the linearization moiety of the P5 primers on the first surface match the linearization moiety of the P7 primers on the second surface, and the linearization moiety of the P7 primers on the first surface match the linearization moiety of the P5 primers on the second surface.

### Nucleic Acids and Template Libraries

As will be understood by the skilled person, a double-stranded nucleic acid will typically be formed from two complementary polynucleotide strands made up of deoxyribonucleotides joined by phosphodiester bonds, but may additionally include one or more ribonucleotides and/or non-nucleotide chemical moieties and/or non-naturally occurring nucleotides and/or non-naturally occurring backbone linkages. In particular, the double-stranded nucleic acid may include non-nucleotide chemical moieties, e.g. linkers or spacers, at the 5′ end of one or both strands. By way of non-limiting example, the double-stranded nucleic acid may include methylated nucleotides, uracil bases, phosphorothioate groups, also peptide conjugates etc. Such non-DNA or non-natural modifications may be included in order to confer some desirable property to the nucleic acid, for example to enable covalent, non-covalent or metal-coordination attachment to a solid support, or to act as spacers to position the site of cleavage an optimal distance from the solid support. A single stranded nucleic acid consists of one such polynucleotide strand. Where a polynucleotide strand is only partially hybridised to a complementary strand—for example, a long polynucleotide strand hybridised to a short nucleotide primer—it may still be referred to herein as a single stranded nucleic.

An example of a typical double-stranded nucleic acid template (which may be provided in a library of such templates) is shown in FIG. 11A. In one example, a first strand of the template includes, in the 5′ to 3′ direction, a first lawn primer-binding sequence (e.g., P5), an index sequence (e.g., i5), a first sequencing primer binding site (e.g., SBS3), an insert corresponding to the template DNA to be sequenced, a second sequencing primer binding site (e.g. SBS12′), a second index sequence (e.g. i7′) and a second lawn primer-binding sequence (e.g. the complement of P7). The second strand of the template includes, in the 3′ to 5′ direction, a first lawn primer-binding site (e.g. the complement of P5), an index sequence (e.g. i5′, which is complementary to i5), a first sequencing primer binding site (e.g. SBS3′ which is complementary to SBS3), an insert corresponding to the complement of the template DNA to be sequenced, a second sequencing primer binding site (e.g. SBS12, which is complementary to SBS12), a second index sequence (e.g. i7, which is complementary to i7) and a second lawn primer-binding sequence (e.g. P7). Either template is referred to herein as a “template strand” or “a single stranded template”. Both template strands annealed together as shown in FIG. 11A, is referred to herein as “a double stranded template”. The combination of a primer-binding sequence, an index sequence and a sequencing binding site is referred to herein as an adaptor sequence, and a single insert is flanked by a 5′ adaptor sequence and a 3′ adaptor sequence. The first primer-binding sequence may also include a sequencing primer for the index read (15).

In one example, the primer-binding sequences of the adaptors are complementary to short primer sequences (or lawn primers) 680, 690 (FIG. 11A) present on the surface 700 (FIG. 11A) of the flow cells. In other examples, the flow cell has a first surface 710 (FIG. 11B) and a second surface 720 (FIG. 11B). Each of the first surface 710 and the second surface 720 contain the short primer sequences (or lawn primers) 680, 690 that are complementary to the primer-binding sequences of the adaptors. Binding of suitable portions of the adaptors to their complements (P5 and P7) on—for example—the surface of the flow cell (FIG. 11A) or on both surfaces of the flow cell (FIG. 11B), permits nucleic acid amplification. As used herein “′” denotes the complementary strand.

The primer-binding sequences in the adaptor which permit hybridisation to amplification (lawn) primers will typically be around 20-40 nucleotides in length, although, in examples, the disclosure is not limited to sequences of this length. The precise identity of the amplification primers, and hence the cognate sequences in the adaptors, are generally not material to the disclosure, as long as the primer-binding sequences are able to interact with the amplification primers in order to direct amplification. The sequence of the amplification primers may be specific for a particular target nucleic acid that it is desired to amplify, but in other examples these sequences may be “universal” primer sequences which enable amplification of any target nucleic acid of known or unknown sequence which has been modified to enable amplification with the universal primers. The criteria for design of PCR primers are generally well known to those of ordinary skill in the art. “Primer-binding sequences” may also be referred to as “clustering sequences” “clustering primers” or “cluster primers” in the present disclosure, and such terms may be used interchangeably.

The index sequences (also known as a barcode or tag sequence) are unique short DNA sequences that are added to each DNA fragment during library preparation. The unique sequences allow many libraries to be pooled together and sequenced simultaneously. Sequencing reads from pooled libraries are identified and sorted computationally, based on their barcodes, before final data analysis. Library multiplexing is also a useful technique when working with small genomes or targeting genomic regions of interest. Multiplexing with barcodes can exponentially increase the number of samples analyzed in a single run, without drastically increasing run cost or run time. Examples of tag sequences are found in WO05068656, the entire contents of which are incorporated by reference herein. The tag can be read at the end of the first read, or equally at the end of the second read. The disclosure is not limited by the number of reads per cluster, for example two reads per cluster: three or more reads per cluster are obtainable simply by dehybridising a first extended sequencing primer, and rehybridising a second primer before or after a cluster repopulation/strand resynthesis step. Methods of preparing suitable samples for indexing are described in, for example U.S. 60/899,221, the entire contents of which are incorporated by reference herein. Single or dual indexing may also be used. With single indexing, up to 48 unique 6-base indexes can be used to generate up to 48 uniquely tagged libraries. With dual indexing, up to 24 unique 8-base Index 1 sequences and up to 16 unique 8-base Index 2 sequences can be used in combination to generate up to 384 uniquely tagged libraries. Pairs of indexes can also be used such that every i5 index and every i7 index are used only one time. With these unique dual indexes, it is possible to identify and filter indexed hopped reads, providing even higher confidence in multiplexed samples.

The sequencing binding sites are sequencing and/or index primer binding sites and indicates the starting point of the sequencing read. During the sequencing process, a sequencing primer anneals (i.e. hybridises) to a portion of the sequencing binding site on the template strand. The DNA polymerase enzyme binds to this site and incorporates complementary nucleotides base by base into the growing opposite strand. In one example, the sequencing process includes a first and second sequencing read. The first sequencing read may include the binding of a first sequencing primer (read 1 sequencing primer) to the first sequencing binding site (e.g., SBS3′) followed by synthesis and sequencing of the complementary strand. This leads to the sequencing of the insert. In a second step, an index sequencing primer (e.g. i7 sequencing primer) binds to a second sequencing binding site (e.g. SBS12) leading to synthesis and sequencing of the index sequence (e.g. sequencing of the i7 primer). The second sequencing read may include binding of an index sequencing primer (e.g. i5 sequencing primer) to the complement of the first sequencing binding site on the template (e.g. SBS3) and synthesis and sequencing of the index sequence (e.g. i5). In a second step, a second sequencing primer (read 2 sequencing primer) binds to the complement of the primer (e.g. i7 sequencing primer) binds to a second sequencing binding site (e.g. SBS12′) leading to synthesis and sequencing of the insert in the reverse direction.

In embodiments described herein, the second sequencing read is not necessary to determine the sequence of the DNA. Data analysis steps subsequent to sequencing can be used to remove the second sequencing read.

Once a double stranded nucleic acid template library is formed, typically, the library has previously been subjected to denaturing conditions to provide single stranded nucleic acids. Suitable denaturing conditions will be apparent to the skilled reader with reference to standard molecular biology protocols (Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al). In one example, chemical denaturation, such as NaOH or formamide, is used. Suitable denaturation agents include: acidic nucleic acid denaturants such as acetic acid, HCl, or nitric acid; basic nucleic acid denaturants such as NaOH; or other nucleic acid denaturants such as DMSO, formamide, betaine, guanidine, sodium salicylate, propylene glycol or urea. Preferred denaturation agents are formamide and NaOH, preferably formamide.

As illustrated in FIGS. 11A and 11B, following denaturation, a single-stranded template library is in one example contacted in free solution onto a solid support comprising surface capture moieties (for example full-length P5 and P7 lawn primers). In some examples, the flow cell has a single surface 700 (FIG. 11A). In some examples, the flow cell has a first surface 710 and a second surface 720 (FIG. 11B). This solid support is typically a flow cell, although in alternative examples, seeding and clustering can be conducted off-flow cell using, for example, microbeads or the like.

### Solid Supports

The disclosure may make use of solid supports made up of a substrate or matrix (e.g. glass slides, polymer beads etc) which has been “functionalised”, for example by application of a layer or coating of an intermediate material comprising reactive groups which permit covalent attachment to biomolecules, such as polynucleotides. Examples of such supports include, but are not limited to, a substrate such as glass. In such examples, the biomolecules (e.g. polynucleotides) may be directly covalently attached to the intermediate material but the intermediate material may itself be non-covalently attached to the substrate or matrix (e.g. the glass substrate). The term “covalent attachment to a solid support” is to be interpreted accordingly as encompassing this type of arrangement. Alternatively, the substrate such as glass may be treated to permit direct covalent attachment of a biomolecule; for example, glass may be treated with hydrochloric acid, thus exposing the hydroxyl groups of the glass, and phosphite-triester chemistry used to directly attach a nucleotide to the glass via a covalent bond between the hydroxyl group of the glass and the phosphate group of the nucleotide.

In other examples, the solid support may be “functionalised” by application of a layer or coating of an intermediate material comprising groups that permit non-covalent attachment to biomolecules. In such examples, the groups on the solid support may form one or more of ionic bonds, hydrogen bonds, hydrophobic interactions, x-x interactions, van der Waals interactions and host-guest interactions, to a corresponding group on the biomolecules (e.g. polynucleotides). The interactions formed between the group on the solid support and the corresponding group on the biomolecules may be configured to cause immobilisation or attachment under the conditions in which it is intended to use the support, for example in applications requiring nucleic acid amplification and/or sequencing. For example, the interactions formed between the group on the solid support and the corresponding group on the biomolecules may be configured such that the biomolecules remain attached to the solid support during amplification and/or sequencing.

In other examples, the solid support may be “functionalised” by application of an intermediate material comprising groups that permit attachment via metal-coordination bonds to biomolecules. In such examples, the groups on the solid support may include ligands (e.g. metal-coordination groups), which are able to bind with a metal moiety on the biomolecule. Alternatively, or in addition, the groups on the solid support may include metal moieties, which are able to bind with a ligand on the biomolecule. The metal-coordination interactions formed between the ligand and the metal moiety may be configured to cause immobilisation or attachment of the biomolecule under the conditions in which it is intended to use the support, for example in applications requiring nucleic acid amplification and/or sequencing. For example, the interactions formed between the group on the solid support and the corresponding group on the biomolecules may be configured such that the biomolecules remain attached to the solid support during amplification and/or sequencing.

Immobilisation or attachment of molecules (e.g. nucleic acids such as positive strands of DNA and negative strands of DNA) to a solid support can be performed through capturing the nucleic acids on the solid support using capture primers. The terms “immobilised” and “attached” are used interchangeably herein and both terms are intended to encompass direct or indirect, covalent or non-covalent attachment, unless indicated otherwise, either explicitly or by context. In certain examples of the disclosure, covalent attachment may be preferred; in other examples, attachment using non-covalent interactions may be preferred; in yet other examples, attachment using metal-coordination bonds may be preferred. However, in general the molecules (e.g. nucleic acids such as positive strands of DNA or negative strands of DNA) remain immobilised or attached to the support under the conditions in which it is intended to use the support, for example in applications requiring nucleic acid amplification and/or sequencing. When referring to attachment of nucleic acids to other nucleic acids, then the terms “immobilised” and “hybridised” are used herein, and generally refer to hydrogen bonding between complementary nucleic acids.

If the amplification is performed on beads, either with a single or multiple extendable primers, the beads may be analysed in solution, in individual wells of a microtitre or picotitre plate, immobilised in individual wells, for example in a fibre optic type device, or immobilised as an array on a solid support. The solid support may be a planar surface, for example a microscope slide, wherein the beads are deposited randomly and held in place with a film of polymer, for example agarose or acrylamide.

### Additional Comments

While various illustrative examples are described above, it will be apparent to one skilled in the art that various changes and modifications may be made therein without departing from the disclosure. The appended claims are intended to cover all such changes and modifications that fall within the true spirit and scope of the disclosure.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

SEQUENCE LISTING

SEQ ID NO: 1
P7 with an 8oxoG linearization site (shown in bold)
/5Hexynyl/TTTTTTCAAGCAGAAGACGGCATAC/8oxoG/AGAT
SEQ ID NO: 2
P5 with a U linearization site (shown in bold)
/5Hexynyl/TTTTTTAATGATACGGCGACCACCGAGAUCTACAC
SEQ ID NO: 3
P15 with a vinyl dT linearization site (shown in bold)
/5Hexynyl/TTTTTTAATGATACGGCGACCACCGAGA/vinyl dT/CTACAC
SEQ ID NO: 4
BCN P15 with a vinyl dT linearization site (shown in bold)
/BCN/TTTTTTAATGATACGGCGACCACCGAGA/vinyl dT/CTACAC
SEQ ID NO: 5
BCN P7 with an 8ox0G linearization site (shown in bold)
/BCN/TTTTTTCAAGCAGAAGACGGCATAC/8oxoG/AGAT
SEQ ID NO: 6
P7 with a U linearization site (shown in bold)
/5Hexynyl/TTTTTTCAAGCAGAAGACGGCATACUAGAT
SEQ ID NO: 7
P5 with an 8oxoG linearization site (shown in bold)
/5Hexynyl/TTTTTTAATGATACGGCGACCACCGAGA/8oxoG/CTACAC
SEQ ID NO: 8
P15 with an 8oxoG linearization site (shown in bold)
/5Hexynyl/TTTTTTAATGATACGGCGACCACCGAGA/8oxoG/CTACAC
SEQ ID NO: 9
BCN P15 with an 8oxoG linearization site (shown in bold)
/BCN/TTTTTTAATGATACGGCGACCACCGAGA/8oxoG/CTACAC
SEQ ID NO: 10
BCN P7 with a vinyl dT linearization site (shown in bold)
/BCN/TTTTTTCAAGCAGAAGACGGCATAC/vinyl dT/AGAT

1. A method of sequencing DNA in a flow cell, comprising:
binding the DNA to a first set of capture primers and to a second set of capture primers,
wherein the first set of capture primers and the second set of capture primers are each coupled to a first surface and to a second surface of the flow cell,
wherein the first set of capture primers on the first surface and the second set of capture primers on the second surface each comprise a first linearization moiety, and
wherein the second set of capture primers on the first surface and the first set of capture primers on the second surface each comprise a second linearization moiety;
linearizing the first linearization moiety or the second linearization moiety, resulting in the DNA remaining coupled to either (i) the first set of capture primers on the first surface and the second set of capture primers on the second surface, or (ii) the second set of capture primers on first surface and first set of capture primers on the second surface;
coupling the DNA bound to either (i) or (ii) with one or more sequencing primers; and
performing at least one sequencing read on the bound DNA, using the one or more sequencing primers.

2. The method of claim 1, wherein the first surface comprises a top surface of the flow cell and the second surface comprises a bottom surface of the flow cell.

3. The method of claim 1, wherein the DNA comprises a first nucleotide sequence on a first end of the DNA that is sufficiently complementary to the first set of capture primers such that the first end and the first set of capture primers are capable of binding to each other, and
wherein the first nucleotide sequence lacks sufficient complementarity with the second set of capture primers, such that the first nucleotide sequence does not bind to the second set of capture primers.

4. The method of claim 1, wherein the DNA comprises a second nucleotide sequence on a second end of the DNA that is sufficiently complementary to the second set of capture primers such that the second end of the DNA and the second set of capture primers are capable of binding to each other, and
wherein the second nucleotide sequence lacks sufficient complementarity to the first set of capture primers, such that the second nucleotide sequence does not bind to the first set of capture primers.

5. The method of claim 3, wherein the first nucleotide sequence and the second nucleotide sequence is on a positive strand of DNA.

6. The method of claim 3, wherein the first nucleotide sequence and the second nucleotide sequence is on a negative strand of DNA.

7. The method of claim 1, further comprising at least one step in which the DNA is amplified in the flow cell.

8. The method of claim 7, wherein the DNA is amplified through bridge amplification or exclusion amplification.

9. The method of claim 1, wherein binding the DNA to a first set of capture primers and to a second set of capture primers, comprises binding a plurality of positive strands and a plurality of negative strands to the first set of capture primers and the second set of capture primers.

10. The method of claim 9, wherein performing the at least one sequencing read comprises performing a first sequencing read and a second sequencing read, wherein the first sequencing read is performed on the plurality of positive strands on the first surface and the second sequencing read is performed on the plurality of negative strands on the second surface.

11. The method of claim 9, wherein performing the at least one sequencing read comprises performing a first sequencing read and a second sequencing read, wherein the first sequencing read is performed on the plurality of negative strands on the first surface and the second sequencing read is performed on the plurality of positive strands on the second surface.

12. A flow cell, comprising:
a first surface comprising a first set of capture primers and a second set of capture primers; and
a second surface comprising a first set of capture primers and a second set of capture primers,
wherein the first set of capture primers on the first surface and the second set of capture primers on the second surface each comprise a first linearization moiety, and wherein the second set of capture primers on the first surface and the first set of capture primers on the second surface each comprise a second linearization moiety.

13. The flow cell of claim 12, wherein the first surface comprises a top surface of the flow cell and the second surface comprises a bottom surface of the flow cell.

14. The flow cell of claim 12, wherein the first linearization moiety comprises any one or more of 8oxoG, U, and vinyl dT, and wherein the second linearization moiety comprises any one or more of 8oxoG, U, and vinyl dT.

15. The flow cell of claim 12, wherein the first set of capture primers comprises at least 80% identity with any one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

16. The flow cell of claim 15, wherein the first set of capture primer comprises any one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

17. The flow cell of claim 15, wherein (i) when the first set of capture primers on the first surface comprises at least 80% sequence identity with SEQ ID NO: 1, the first set of capture primers on the second surface comprises at least 80% sequence identity with SEQ ID NO: 6, (ii) when the first set of capture primers on the first surface comprises at least 80% sequence identity with SEQ ID NO: 2, the first set of capture primers on the second surface comprises at least 80% sequence identity with SEQ ID NO: 7, (iii) when the first set of capture primers on the first surface comprises at least 80% sequence identity with SEQ ID NO: 3, the first set of capture primers on the second surface comprises at least 80% sequence identity with SEQ ID NO: 8, (iv) when the first set of capture primers on the first surface comprises at least 80% sequence identity with SEQ ID NO: 4, the first set of capture primers on the second surface comprises at least 80% sequence identity with SEQ ID NO: 9, or (v) when the first set of capture primers on the first surface comprises at least 80% sequence identity with SEQ ID NO: 5, the first set of capture primers on the second surface comprises at least 80% sequence identity with SEQ ID NO: 10.

18. The flow cell of claim 15, wherein (i) the first set of capture primers on the first surface comprises SEQ ID NO: 1 and the first set of capture primers on the second surface comprises SEQ ID NO: 6, (ii) the first set of capture primers on the first surface comprises SEQ ID NO: 2 and the first set of capture primers on the second surface comprises SEQ ID NO: 7, (iii) the first set of capture primers on the first surface comprises SEQ ID NO: 3 and the first set of capture primers on the second surface comprises SEQ ID NO: 8, (iv) the first set of capture primers on the first surface comprises SEQ ID NO: 4 and the first set of capture primers on the second surface comprises SEQ ID NO: 9, or (v) the first set of capture primers on the first surface comprises SEQ ID NO: 5 and the first set of capture primers on the second surface comprises SEQ ID NO: 10.

19. The flow cell of claim 12, wherein the second set of capture primers comprises at least 80% identity with any one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

20. The flow cell of claim 19, wherein the second set of capture primers comprises any one or more of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10.

21-22. (canceled)
